(19) United States
(12) Patent Application Publication
(10) Pub. No.: US 2007/0224593 A1
(43) Pub. Date: Sep. 27, 2007

(54) Diagnosis of transmissible spongiform encephalopathy

(76) Inventors: Lee E. Goldstein, Wellesley, MA (US); Katherine I. O'Rourke, Pullman, WA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/583,319

(22) Filed: Oct. 18, 2006

Related U.S. Application Data
(60) Provisional application No. 60/727,917, filed on Oct. 18, 2005.

(51) Int. Cl. C12Q 1/70; G01N 33/53

(52) U.S. Cl. 435/5; 435/7.1

(57) ABSTRACT

The invention features a method of diagnosing or providing a prognosis regarding the state of Transmissible Spongiform Encephalopathies (TSEs) in a mammal by contacting a target tissue or other environmental sample with a detectable compound, which binds to a non-amyloid form of a disease specific prion (PrP-d). An increase in binding of the compound to the target tissue or environmental sample compared to a normal control level of binding indicates that the mammal is suffering from or is at risk of developing TSE, or that the sample is contaminated with TSE-infected material.

### RELATED APPLICATIONS

This application claims priority to U.S. Provisional Application No. 60/727,917, filed Oct. 18, 2005, the contents of which are hereby incorporated by reference in their entirety.

### STATEMENT AS TO FEDERALLY SPONSORED RESEARCH

This invention was made with U.S. Government support from the United States Department of Agriculture, Agricultural Research Service (CRIS 5348-32000-021-00D). The government has certain rights in the invention.

### TECHNICAL FIELD

This invention relates to neurodegenerative disease.

### BACKGROUND OF THE INVENTION

Transmissible Spongiform Encephalopathies (TSEs) are prion diseases characterized by fatal spongiform neurodegeneration of the brain and associated with severe and fatal neurological signs and symptoms. TSE prionopathies that occur in humans include Creutzfeld-Jacob Disease (CJD); new variant, Creutzfeld-Jacob Disease (nv-CJD); Gertsmann-Straussler-Scheinker syndrome; fatal familial insomnia; Kuru; and Alpers Syndrome. Bovine Spongiform Encephalopathy (BSE) is one of several different forms of TSE affecting a number of animal species. Scrapie is a common disease in sheep and goats. Chronic wasting disease (CWD) of deer and elk (cervid animals) is another TSE disease that occurs in wild and farmed cervid animals. TSEs also occur in minks, cats and other animals.

Cattle, sheep, deer, elk and goat herds are currently monitored for TSEs by testing dead and slaughtered animals. Sheep, deer, elk and goats are also tested by ante-mortem tissue biopsy. However, the current tests have major drawbacks.

### SUMMARY

The invention provides methods for reliable detection of TSE or a pre-morbid neurodegenerative state early in the infection process, and throughout the course of the disease. The diagnostic method is carried out by contacting a target tissue or target fluid of a mammal with a detectable compound which binds to a disease specific, proteinase K resistant prion protein (PrP-d). For example, a non-central nervous system (CNS) tissue of a mammal, e.g., a bovine, sheep, rodent, mink, cervid or human subject, is contacted with a detectable compound that binds to PrP-d. Preferably, the detectable compound is not a peptide and does not contain an antibody or fragment thereof. For example, the detectable compound is a small molecule fluorophore that binds to PrP-d. There are two isoforms of disease specific prion proteins, which are known as amyloid PrP-d and non-amyloid PrP-d. Isoforms of non-amyloid PrP-d are also referred to as pre-amyloid PrP-d.

Classical amyloid PrP-d has a beta-pleated sheet secondary structure and collects as extracellular deposits of amyloid fibrils. These amyloid PrP fibrils contain double protein helices of 1000 angstrom periodicity consisting of two beta pleated sheet micelles in the form of twin filaments separated by an interspace. These fibrils also demonstrate characteristic electron microscopy (EM) appearance (e.g., masses of tangled, extracellular, unbranched filaments that are usually arranged in random orientation, where each fibril is approximately 8-10 nm diameter and has a variable length, e.g., up to several millimicrons in length) that conform to the properties observed in other classical amyloid proteins (such as β-amyloid, Aβ, in Alzheimer's disease). (See Caughey and Lansbury, Jr., Annu. Rev. Neurosci. vol. 26:267-298 (2003)). As is true of other amyloid proteins, amyloid PrP-d has an affinity for the dye Congo red, i.e., the sodium salt of benzidinediazo-bis-1-naphtylamine-4-sulfonic acid binds within the groove face of the amyloid fibril and exhibits characteristic apple green birefringence under intense cross-polarized light. Non-amyloid (or pre-amyloid) PrP-d is a globular proteinaceous aggregate characterized by thioflavin T (ThT) and 1-anilino-8-naphthalenesulfonate (ANS) binding and a beta-sheet-rich structure. ThT is known to interact with the beta-sheet quaternary structure (see e.g., Levine, Amyloid vol. 2:10 (1995)), and ANS is a fluorescent probe that is used to measure protein unfolding as evidenced by increased hydrophobicity.

Amyloid PrP-d and non-amyloid PrP-d are distinguished on the basis of their optical properties and structural differences. Amyloid, but not pre/non-amyloid, is specifically characterized by Congophilia and apple-green birefringence at the light microscope level, and characteristic extracellular fibrils at the ultrastructural levels. Both amyloid and pre/non-amyloid are resistant to proteinase K digestion. In contrast to amyloid PrP-d, non-amyloid PrP-d is an amorphous globular aggregate matrix that may contain embedded protofibrillar structures. In contradistinction to amyloid PrP-d, non-amyloid PrP-d does not contain clearly defined classical amyloid fibrils and does not exhibit apple-green birefringence after binding to Congo Red and exposure to intense cross-polarized light illumination.

According to a consensus statement by experts in the field of amyloid research, PrP-d accumulations that do not possess these distinctive amyloid characteristics, e.g., filamentous EM appearance, Congo Red binding, and apple green birefringence following contact with Congo Red and cross-polarized light illumination, by definition, are non-amyloid PrP-d. (see e.g., Westermark, P., et al., “Amyloid fibril protein nomenclature—2002”, Amyloid, 9:197-200 (2002)). As used herein, the terms “non-amyloid” and “pre-amyloid” encompass all PrP-d that do not exhibit the classical amyloid characteristics of amyloid PrP-d described above.

The methods described herein are used to target and detect the presence of non-amyloid PrP-d disease component, as distinguished from the classical amyloid form of the protein.

The detectable compounds used in the methods described herein bind to a disease specific prion protein or collection of prion protein particles. For example, the compound preferentially binds to a pre/non-amyloid form, e.g., the compound binds at least 10%, 25%, 50%, 75%, 2-fold, 5-fold, 10-fold or more compared to the level of binding of the compound to an amyloid form of the prion protein. For example, the compound binds to the protofibrils of the non-amyloid isoform or another fragment of a disease specific prion protein.

The methods described herein are used to detect PrP-d, e.g., non-amyloid PrP-d, in a variety of target tissues, such as lymphatic and para-lymphatic tissues such as, e.g., retropharyngeal lymphatic tissue, parotid lymphatic tissue, sentinel lymphatic tissue, axillary lymphatic tissue, inguinal lymphatic tissue and peripheral lymphatic tissues including gut associated lymphatic tissue, rectal mucosal associated lymphatic tissue, nictitating membrane associated lymphatic tissue, mammary gland associated lymphatic tissue, and placenta, or a variety of target fluids. Suitable tissues and fluids also include blood, serum, saliva, amniotic fluid, chorionic villi, eye tissue (e.g., lens, retina), nictitating membrane, brain tissue, urine, tears, mucous, mucous membrane, cerebrospinal fluids and feces. The target tissue or target fluid samples also include samples that are derived from a target tissue or target fluid, such as, for example, a protein lysate sample or other processed samples, such as, e.g., the white blood cell compartment (i.e., buffy coat), isolated B cells, isolated T cells, CD4+ cells, CD8+ cells, of a processed, e.g., centrifuged, whole blood sample. An increase in binding of the compound to a target tissue, e.g., peri-ocular lymphatic tissue or retropharyngeal lymphatic tissue, or target fluid, e.g., blood, serum, or buffy coat, compared to a normal control level of binding indicates that the mammal is suffering from, has been exposed to, is infected with, is a carrier for, or is at risk of developing TSE.

The diagnostic methods described herein are carried out ante-mortem, postmortem or both. These methods are used in the diagnosis, prognosis, detection and profiling of TSE in a subject, and these methods are also used to assess a subject's risk of developing TSE. When the methods are carried out on a living subject, the detectable compound is administered (e.g., intravenously, intraperitoneally, topically) in the living subject, and then extracorporeal imaging, e.g., infrared imaging, is used to detect and quantify the level of fluorescence in the target tissue or fluid site. Alternatively, a tissue or fluid sample is taken from the living subject and analyzed ex vivo. The methods described herein are also carried out on fetal subjects, fetal tissue-derived or fetal fluid-derived samples, such as for example, amniotic fluid and chorionic villi samples, in order to determine whether offspring is suffering from, has been exposed to, is infected with, is a carrier for, or is at risk of developing TSE. The tissue or fluid sample is analyzed in utero or in vitro.

The detectable compound, also referred to herein as a probe or agent, is, for example, a small molecule fluorophore. Preferably, the detectable compound is not a peptide, polypeptide, protein or an antibody, e.g., the detectable compound is not a detectably labeled polypeptide. For example, the detectable compound is a detectable methoxy agent such as Me-X04 (1,4-bis(4′-hydroxystyrl)-2-methoxybenzene) (FIG. 1). Me-X04 has an excitation maximum of 368 nm (±20 nm) and an emission maximum of 450 nm (±20 nm) (FIG. 4). Another detectable methoxy agent is X34 (1,4-bis(3-carboxy-4-hydroxyphenylethenyl)-benzene) (FIGS. 2 and 3).; Other methoxy agents include, e.g., Chrysamine or Chrysamine derivative compound such as {(trans, trans),-1-bromo-2,5-bis-(3-hydroxycarbonyl-4-hyrdoxy)styrlbenzene (BSB)}. The structure of Chrysamine G is provided in FIG. 2, and several Chrysamine G derivatives are shown in FIG. 3 (see e.g., Mathis et al., Curr. Pharm. Des., vol. 10(13):1469-93 (2004); U.S. Pat. Nos. 6,417,178; 6,168,776; 6,133,259; and 6,114,175, each of which is hereby incorporated by reference in its entirety). The detectable compound is not a nonspecific amyloidphilic probe such as, for example, thioflavin T, thioflavin S or Congo red dye.

Binding of detectable probe agents such as Me-X04 or X-34 to non-amyloid or pre-amyloid PrP-d was a surprising result, because prior to the invention such compounds were believed to associate only with classical amyloid PrP-d isoforms.

The detectable compounds used in the methods described herein are intrinsically fluorescent (i.e., the molecule itself is capable of fluorescing when excited with light of the appropriate wavelength and, therefore, does not require the addition of a detectable chemical tag to fluoresce). The fluorescence profile of the Me-X04 agent demonstrates the following spectral maxima: wavelength excitation peak at about 368 nm (excitation wavelength λex) and emission peak at about 450 nm (emission wavelength, λem) (FIG. 4). Detectable compounds used in the methods described herein emit at a wavelength less than 475 nm, preferably less than 460 nm and most preferably in the range of 450 nm. Other detectable compounds for use in the methods described herein are intrinsically fluorescent and emit at a wavelength greater than 600 nm, e.g., λem in the range of 600-800 nm, λem in the infrared range. For example, suitable detectable compounds exhibit fluorescence profiles specific for each detection agent, preferably with an λex/λem profile in the range of 300-1000 nm.

In the methods of the invention, the detectable compounds are excited with any illumination source, such as for example quasi-electric light, filtered light, laser light or any light source having a specific wavelength for fluorescent excitation that correlates to the fluorescence profile of the detectable compound(s) being used. Suitable illumination sources include, for example, low wattage infrared laser light (also referred to as a low intensity infrared laser).

These detectable compounds are used to identify and analyze the presence of non-amyloid PrP-d in a target tissue, target fluid or other sample. The ability to detect non-amyloid isoform of PrP-d is an advantage over methods that are limited to the detection of amyloid PrP-d. For example, the amyloid form of PrP-d is not present in all disease states, and furthermore, some species, e.g., sheep, rarely exhibit amyloid PrP-d accumulation, even though the subject is infected. Thus, unlike methods that detect only the presence of amyloid PrP-d, the methods described herein are used to detect TSE, even when amyloid PrP-d is not present, such as in an infected animal that does not typically produce amyloid PrP-d accumulation, or at a stage before amyloid PrP-d is produced or accumulated in the tissue of a subject.

The methods permit detection of the non-amyloid PrP-d isoform at very early stages of infection and throughout the course of the disease, e.g., 1, 2, 3, 4, 5, 6, 7, 8, 9, 10, 11, 12, 16, 20, 24, 28, 32, 36, 40, 44, 48 or 52 weeks or 1, 2, 3, 4, 5, 6 or 7 years after exposure. For example, pre/non-amyloid PrP-d in accordance with the methods described herein is detected prior to 42 days in mule deer and prior to 2 months after a suspected or confirmed date of exposure to an infectious agent and at least 24 months prior to detection of clinical disease as measured by behavioral and motor changes. Those skilled in the art will appreciate that this time frame is dependent upon the species and tissue that is being tested, as well as the detectable agent that is being used to detect non-amyloid PrP-d.

The methods are useful for minimally invasive or non-invasive in vivo screening to identify subjects that have deposits of PrP-d in a variety of target tissues and target fluids, e.g., non-CNS tissues such as the lymphatic tissue, blood and ocular tissue, for pre-morbid neurodegenerative state, diagnosis, prognosis, and monitoring patient responses to drug therapy for TSE. In these in vivo methods, a target tissue or fluid of a test subject is contacted with a detectable compound, allowed to penetrate cells in the tissue or fluid, and specific detectable signal from the compound is detected using standard methods (e.g., using a photon detector such as a photomultiplier chip) upon exposure of the target tissue or target fluid to fluorescence excitation (e.g., illumination with light in the appropriate excitation wavelength range), as measured using standard methods, e.g., fluorescence scanning. The detectable compound is applied directly to the target tissue or fluid, or, alternatively, the detectable compound is administered (e.g., intravenously, intraperitoneally, intradermally, topically) to a subject.

In other methods, the target tissue or target fluid is analyzed in vitro. For example, a target tissue of a test subject is contacted with a detectable compound, allowed to penetrate cells in the sample, the sample tissue is removed from the subject, e.g., by biopsy, blood drawing or other surgical extraction, and the level of fluorescence is measured using standard methods, e.g., fluorescence microscopy or fluorescence plate reader. For blood samples drawn from the subject, the sample is analyzed as whole blood, e.g., by examining the whole blood using fluorescent microscopy, or, alternatively, the blood sample is processed before fluorescence analysis, e.g., by spinning the blood sample down and examining the white blood cell fraction (i.e., buffy coat), using fluorescent microscopy, fluorescence-activated cell sorter (FACS) or by fluorescence polarization, in which contact between the sample and the detectable compound results in a change in rotation of the compound in solution. Detectable methoxy agents, such as, for example, X-34, are used in fluorescence polarization imaging. Other suitable methods of fluorescence analysis include, for example, direct/indirect fluorescence, fluorescence spectroscopy, fluorescence autocorrelation spectroscopy, fluorescence polarization, multiplex fluorescence with/without ratiometry, fluorescence resonance energy transfer (FRET), quantum dot/nanoparticle fluorescence (with/without multiplexing), confocal fluorescence, multiphoton/multi-pulse/multiband fluorescence, fluorescence lifetime imaging, time-resolved fluorescence, and fluorescence recovery after photobleaching (FRAP). These fluorescence techniques are used at any point in the imaging process. These fluorescence techniques are used alone or in any combination thereof. These fluorescence techniques are also used in combination with other scattering and/or spectroscopic techniques.

Blood, blood components, bodily fluids and other washings from fecal materials or bodily openings such as the nasal cavities are, for example, applied to a solid support, either directly or after enrichment for PrP-d, and contacted with the detectable compound. The detectable compound is applied directly to the target tissue or fluid, or, alternatively, the detectable compound is administered systemically, locally or topically to a subject. Fluorescence polarization is performed in the fluid phase. Thus, in fluorescence polarization, a fluid sample, such as blood, blood components, bodily fluids and other washings from fecal materials or bodily openings, e.g., the nasal cavities is, either directly or after enrichment for PrP-d, contacted with the detectable compound. In fluorescence polarization, the detectable compound is applied directly to the fluid sample, or, alternatively, the detectable compound is administered systemically, locally or topically to a subject. These in vitro analytical methods are conducted ante-mortem, post mortem or both.

An increase of at least 10% compared to fluorescence of a normal control subject (after probe administration) or a 10% decrease in the rotation of the probe as detected by fluorescence polarization indicates TSE or a predisposition thereto. Preferably, the difference is 20%, 50%, 75%, 100%, three-fold, ten-fold or more compared to a normal control value. A diagnosis of TSE or a predisposition thereto is also indicated when the level of fluorescence detected in a subject is at least, e.g., one, two or three, standard deviations above the mean level of fluorescence for a population of normal control subjects. A normal control value typically corresponds to little or no binding of the probe to the target tissue or fluid. The level of normal target tissue or fluid fluorescence is the level of fluorescence detected after contacting target tissue or fluid of a normal, TSE-free subject (or population of subjects) with a PrP-d-binding detectable compound. The value is optionally derived by determining the average or mean of values derived from a pool of individual subjects known to be free of TSE (as well as free of known genetic predisposition thereto). If the probe used emits light in the range of normal target tissue or target fluid autofluorescence, the level of autofluorescence is factored into the reading. For example, a 10% increase in fluorescence (after probe administration) compared to the level in the absence of the probe (autofluorescence) indicates a pathological state or predisposition to developing a neuropathological state. Preferably, baseline autofluorescence is established (prior to probe administration) for each individual. A diagnostic level of fluorescence is preferably at least 25%, more preferably at least 50%, more preferably at least 100% greater than a normal control value. For example, detection of fluorescence from a non-amyloid PrP-d-specific detectable compound that is 2-fold or more greater than a normal control value (e.g., 2 fold, 2.5 fold, 5 fold, 10 fold), indicates a pathological state.

A method for prognosis of TSE includes the steps of (a) contacting a target tissue-derived or target fluid-derived sample of a mammal with a detectable compound which binds to a disease-specific prion protein (b) quantitating binding of the compound to the sample; and (c) comparing the level of binding with a normal control level of binding. Increased levels of binding over time indicates an adverse prognosis. Target tissue or fluid fluorescence in the test subject after compound administration is compared to endogenous autofluorescence of a non-TSE subject (or population of individuals) or the level of fluorescence of a non-TSE subject (or population of non-TSE subjects) after probe administration. The methods are also used to stage severity of disease, monitor responses to drug treatment, and screen drugs for the ability to inhibit PrP-d accumulation. An increased level of fluorescence (indicative of PrP-d accumulation in the target tissue or fluid) indicates a more advanced stage of TSE. A reduction in the level of fluorescence (indicative of PrP-d accumulation in the target tissue or fluid) over time indicates that a given drug inhibits PrP-d accumulation and indicates a positive clinical response to drug treatment. A decrease in the rotation of the probe as detected by fluorescence polarization indicates a more advanced stage of TSE. A reduction in the level of decreased rotation of the probe over time indicates that a given drug inhibits PrP-d accumulation and indicates a positive clinical response to drug treatment.

The methods described herein are also used to detect the presence of non-amyloid PrP-d in a variety of environmental samples. For example, the methods are used as a means of surveillance for suspected contaminated surfaces, e.g., slaughtering surfaces, butchering surfaces, feeding surfaces, and grazing surfaces. Other environmental surfaces that are susceptible to being contaminated include, for example, hospital surfaces such as surgical instruments, operating surfaces and examination surfaces. The risk of contamination is present in surgical procedures, which involve contact with tissues in which prions are known to accumulate. For example, the risk of contamination is present in tonsillectomies and appendectomies, which involve contact with lymphatic tissue, as prions are now known to accumulate in lymphatic tissue. During such surgical procedures, the methods described herein are used to screen for possible contamination of exposed surfaces and surgical instruments. It is a clinical reality that disposable instruments are not feasible for use in these operations, and, therefore, surgeons use reusable instruments, particularly for children. Thus, the methods described herein help prevent inadvertent patient-to-patient transmission.

To test surfaces that are suspected or susceptible to being contaminated, a swipe test is used, in which the suspected surface is contacted with a detectable agent that recognizes non-amyloid PrP-d, e.g., Chrysamine G or a derivative thereof, such as the agent Me-X04 (1,4-bis (4′-hydroxystyrl)-2-methoxybenzene), and the surface is washed to remove any unbound agent. The level of fluorescence on the suspected surface is then imaged under a fluorescent light source.

Other environmental samples that are used in the methods described herein to detect the presence of PrP-d include environmental fluids such as water. Fluid that is suspected of being contaminated is passed through a filter (e.g., pore size 0.2 microns), and contacted with a detectable agent. The level of fluorescence in the flow-through fluid sample or on that associated with compositions retained on the filter is then imaged under a fluorescent light source. Alternatively or in addition, the environmental fluid sample is analyzed using fluorescence polarization in which the fluid sample is, either directly or after enrichment for PrP-d, contacted with the detectable compound. The change in rotation of the compound in solution after contact with the fluid sample is then detected and analyzed. Alternatively, the fluid sample is added to a solid glass or plastic reservoir, e.g., a plastic microtiter plate, and the detectable agent is added to the sample. The reservoir is washed to remove any unbound agent and then imaged under a fluorescent light source.

The methods described herein are also used to detect the presence of PrP-d in environmental samples such as soil and other land materials, including vegetation such as plants, crops, trees and groundcover. PrP-d is analyzed by contacting the land material directly with the detectable agent and washing the sample to remove any unbound agent. The level of fluorescence in the suspected material is then imaged under a fluorescent light source. Alternatively, the land material that is suspected of being contaminated is washed in laboratory-grade water, and the water is collected, passed through a filter, contacted with a detectable agent and washed to remove any unbound agent. The level of fluorescence in the water that was used to contact the suspected land material is then imaged under a fluorescent light source. These land materials are analyzed at a variety of progressive increments to determine the extent of TSE contamination. For example, when detecting the presence of PrP-d in dirt or other soil samples, the suspected samples are taken from various depths, e.g., 0.1, 0.25, 0.5, 1, 5, and 10 inches, to determine how far the contamination extends below the surface. Such methods are useful, e.g., to test the soil and vegetation in areas where animals are known to be, or suspected to be, suffering from TSE as a means to determine whether healthy (i.e., uninfected) animals should be allowed to graze, inhabit or otherwise come in contact with the suspected area of land.

The detectable compounds, e.g., polypeptide ligands, organic compounds, or inorganic compounds, are isolated or purified. An “isolated” or “purified” composition is substantially free of cellular material or other contaminating proteins from the cell or tissue source from which it is derived, or substantially free from chemical precursors or other chemicals when chemically synthesized. Preferably, a preparation of a compound, e.g., a fluorescent PrP-d-binding compound, is at least 75%, more preferably 80%, more preferably 85%, more preferably 90%, more preferably 95%, more preferably 98%, and most preferably 99 or 100% of the dry weight of the preparation.

“Fluorescence” is the phenomenon in which light energy (“exciting light”) is absorbed by a molecule resulting in the molecule becoming “excited.” After a pre-described interval such as 1 minute-24 hours, the absorbed light energy is emitted by the excited molecule. The wavelength of the emitted light is typically at a longer wavelength than the exciting light. This emitted light is referred to as fluorescent light. A molecule that exhibits fluorescence is referred to as a “fluorophor.” The relationship between wavelengths of light and degree of excitation of a given fluorophor at that wavelength is described by the “excitation spectrum” of the fluorophor. The excitation spectrum is also called the excitation wavelength range. The relationship between the wavelength of light and the intensity of the fluorescence emission at that wavelength is described by the emission spectrum or fluorescence spectrum of the fluorophor. The emission spectrum is also called the emitted wavelength range. The excitation maximum is the wavelength of exciting light at which fluorescence of the fluorophor reaches maximum intensity. The emission maximum is the wavelength of light emitted by the excited fluorophor when its fluorescence is at maximum intensity.

Most fluorophors excited by and emitting visible light have an emission spectrum overlapping their excitation spectrum, although the maximum for each is different. The distance in nanometers between the excitation spectrum maximum and the emission spectrum maximum is known as the “Stokes' shift.” Fluorophors with large Stokes' shifts in the visible range work best in methods described herein. For example, a fluorophor with an excitation maximum of 400 nm and an emission maximum of 700 nm with little or no overlap between the spectra are used in the methods described herein.

The methods described herein offer several advantages over existing approaches to TSE diagnosis, which use antibodies that recognize various amyloid targets. For example, the method provides non-invasive or minimally invasive ante-mortem diagnosis that accurately and reliably identifies PrP-d accumulation in living tissues and fluids. Prior to the invention, detection methods used neurological or lymphatic tissue samples from deceased subjects, such as slaughtered bovines, ovines and cervid subjects.

Additionally, the method provides rapid and accurate postmortem analysis of tissue and fluid samples. In addition, the method utilizes physiologically-compatible probes. Moreover, the scanning procedure itself takes a matter of seconds, e.g., 30 seconds to a few minutes. Furthermore, the methods described herein detect non-amyloid PrP-d. In particular, non-amyloid PrP-d accumulates in an unique anatomical pattern in target tissues and fluids such as lymphatic tissue, ocular tissue and blood, because these tissues and fluids exhibit little or no significant non-amyloid PrP-d accumulation in a non -diseased stage. Thus, even small amounts of non-amyloid PrP-d prion accumulation are easily detectable in target tissues and fluids such as lymphatic tissues (e.g., such as peri-ocular lymphatic tissue, parotid lymphatic tissue, sentinel lymphatic tissue, axillary lymphatic tissue, inguinal lymphatic tissue, peripheral lymphatic tissues, and retropharyngeal lymphatic tissue, gut associated lymphatic tissue, rectal mucosa associated lymphatic tissue, supramammary lymphatic tissue, lymphatic follicles in the mammary gland, ocular tissue, placenta, feces, urine, tears, saliva, and blood.

Other features, objects, and advantages of the invention will be apparent from the description and drawings.

### BRIEF DESCRIPTION OF THE DRAWINGS

FIG. 1 is an illustration depicting the structure of the detectable compound Me-X04.

FIG. 2 is an illustration depicting the structure of Chrysamine G and derivatives thereof.

FIG. 3 is an illustration depicting the structure of Chrysamine G and derivatives thereof.

FIG. 4 is a graph depicting the excitation and emission spectra of the detectable compound Me-X04.

FIGS. 5A and 5B are photomicrographs showing scrapie-form prion protein (PrP-d) detection and visualization in TSE⁺ sheep. Retropharyngeal lymph nodes were analyzed ex vivo. TSE^{±} tissue immersed in Me-X04 (100 μM, 10 min, RT), differentiated (alkaline EtOH 50%, pH 10), rinsed, and imaged (Optiscan 2400, Neuroptix), operating in fluorescence imaging mode with optical bandpass filters centered on excitation wavelength (λex) of approximately 370 nm, emission wavelength (λem) of approximately 450 nm. The exposures (500 msec) and post-exposure thresholding were identical. TSE status was confirmed by PrP-d immunohistochemistry.

FIGS. 6A and 6B are photographs depicting the correlation of imaging using the X-34 methoxy agent (Panel A) and conventional immunohistochemistry (Panel B) on the same section of unfixed lymphoid tissue from scrapie positive sheep. The arrows in Panel A and Panel B illustrate the colocalization of the X34 binding and the antibody binding, as detected by AEC (3-Amino 9-ethylcarbazole.).

### DETAILED DESCRIPTION

Prion diseases are transmissible, conformational disorders manifesting as a rapidly progressing dementia invariably leading to death. The prionopathies are characterized by accumulation of a disease-specific prion such as the toxic insoluble disease-specific conformer scrapie-form prion protein (PrP-d, also referred to as PrP^{Sc} or PrP^{res}) of the normally expressed cellular prion protein (PrP^{C}). The relatively short symptomatic illness, e.g., days to months, is preceded by a period, e.g., more than a year, typically 3 to 5 years, during which time PrP-d accumulates in various organs outside the central nervous system (CNS). The invention provides methods for detecting PrP-d, such as PrP^{Sc}, in a target tissue or fluid, e.g., a non-CNS tissue, as an early marker of prion infection. The methods are useful to screen wild and domesticated animals as well as humans who are asymptomatic carriers of prion infection and would thus constitute a threat to recipients of organ transplantation or blood products. The test identifies prion-infected animals and is useful to cull populations to prevent infected animals from entering the human food chain.

The methods are used to detect the presence of PrP-d, such as non-amyloid PrP-d and pre-amyloid PrP-d. There are two categories of disease specific prions, amyloid PrP-d and non-amyloid PrP-d. The term “amyloid” refers to a form of protein that possesses a variety of distinctive characteristics, as established by the International Society of Amyloidosis (see e.g., Westermark, P., et al., Amyloid fibril protein nomenclature—2002. Amyloid, 9:197-200 (2002), hereby incorporated by reference in its entirety). Classical amyloid PrP-d has a beta-pleated sheet secondary structure and collects as extracellular deposits of amyloid fibrils. These amyloid PrP fibrils contain double protein helices of 1000 angstrom periodicity consisting of two beta pleated sheet micelles in the form of twin filaments separated by an interspace. These fibrils also demonstrate characteristic electron microscopy (EM) appearance (e.g., masses of tangled, extracellular, unbranched filaments that are usually arranged in random orientation, where each fibril is approximately 8-10 nm diameter and has a variable length, e.g., up to several millimicrons in length) that conform to the properties observed in other classical amyloid proteins (such as β-amyloid, Aβ, in Alzheimer's disease). (See Caughey and Lansbury, Jr., Annu. Rev. Neurosci. vol. 26:267-298 (2003)). As is true of other amyloid proteins, amyloid PrP-d has an affinity for the dye Congo red, i.e., the sodium salt of benzidinediazo-bis-1-naphtylamine-4-sulfonic acid binds within the groove face of the amyloid fibril and exhibits characteristic apple green birefringence under intense cross-polarized light.

Non-amyloid PrP-d, in contrast, is a globular proteinaceous aggregate characterized by thioflavin T (ThT) and 1-anilino-8-naphthalenesulfonate (ANS) binding and a beta-sheet-rich structure. ThT is known to interact with the beta-sheet quaternary structure (see e.g., Levine, Amyloid vol. 2:10 (1995)), and ANS is a fluorescent probe that is used to measure protein unfolding as evidenced by increased hydrophobicity.

The methods described herein offer several advantages over existing approaches to TSE diagnosis, which use antibodies that recognize various amyloid targets. First, the methods permit minimally invasive or non-invasive, ante-mortem diagnosis that accurately and reliably identifies PrP-d accumulation in living tissues, e.g., non-CNS tissues at early stages of infection and throughout the course of the disease. These methods also provide rapid postmortem diagnosis that accurately and reliably identifies PrP-d accumulation in tissues or fluids. Additionally, the methods described herein do not depend on the presence of amyloid PrP-d in order to accurately and reliably diagnose TSE in a subject. The amyloid form of PrP-d is not present or associated with all prion-related disease states; moreover, some species, such as sheep do not typically exhibit significant amyloid PrP-d production or accumulation, even though the animal is infected with TSE. Thus, unlike methods that detect only the presence of amyloid PrP-d, the methods described herein reliably detect TSE, even when amyloid PrP-d is not present.

The methods identify prion infection and TSE before the development of plaques or other amyloid aggregation and before development of overt clinical symptoms such as physical impairment (e.g., difficulty moving, lack of coordination, unsteady gait) and mental impairment (e.g., dementia, changes in social behavior).

Diagnostic Probes and Methods and Methods

The diagnostic probe or imaging agent described herein have the following characteristics: (1) they bind PrP-d even in the non-amyloid isoform; (2) they bind PrP-d selectively and not PrP-c in the same tissue; and (3) they have intrinsic fluorescence.

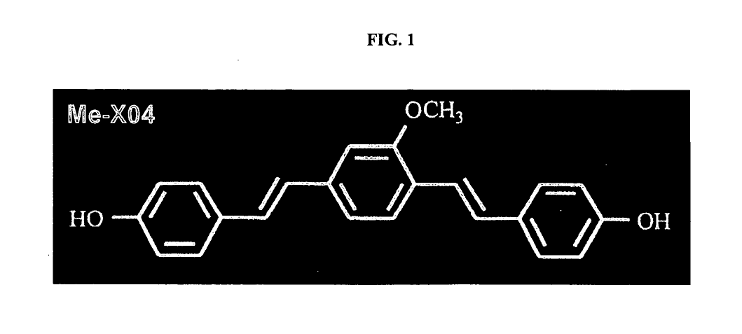

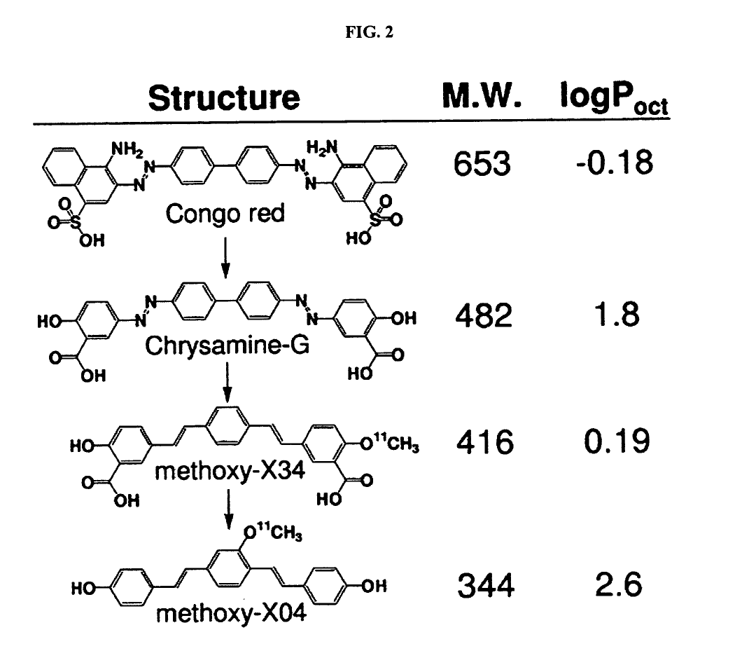

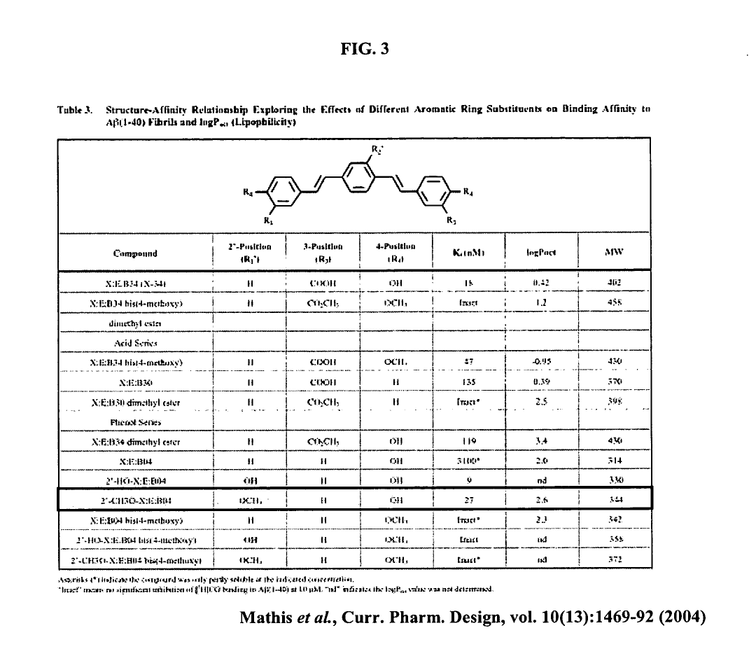

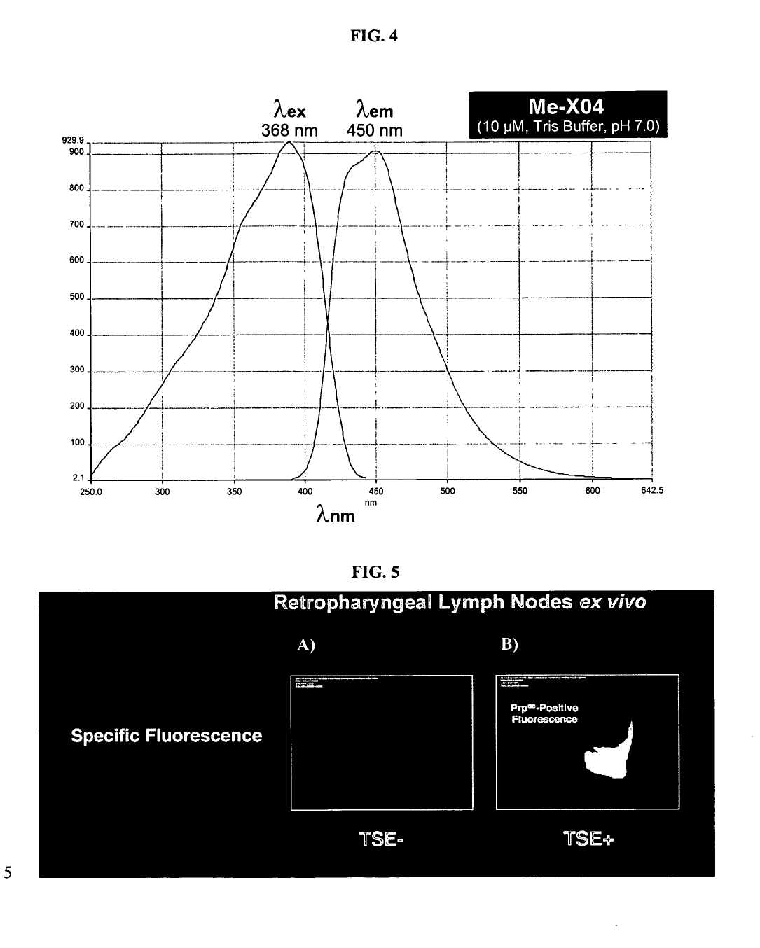

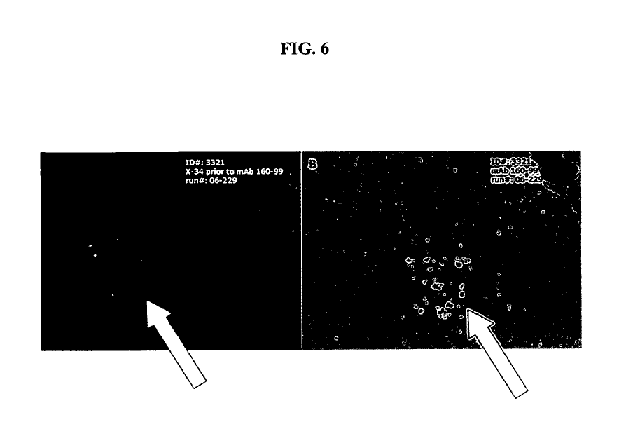

The detectable compound, also referred to herein as a probe or agent, is, for example, a small molecule fluorophore. Preferably, the detectable compound is not a peptide, polypeptide, protein or an antibody, e.g., the detectable compound is not a detectably labeled polypeptide. For example, the detectable compound is a detectable methoxy agent such as Me-X04 (1,4-bis (4′-hydroxystyrl)-2-methoxybenzene) (FIG. 1). Me-X04 has an excitation maximum of 368 nm (±20 nm) and an emission maximum of 450 nm (±20 nm) (FIG. 4). Other methoxy agents include, e.g., Chrysamine or Chrysamine derivative compound such as {(trans, trans),-1-bromo-2,5-bis-(3-hydroxycarbonyl-4-hyrdoxy)styrlbenzene (BSB)}. The structure of Chrysamine G is provided in FIG. 2, and several Chrysamine G derivatives are shown in FIG. 3 (see e.g., Mathis et al., Curr. Pharm. Des., vol. 10(13):1469-93 (2004); U.S. Pat. Nos. 6,417,178; 6,168,776; 6,133,259; and 6,114,175, each of which is hereby incorporated by reference in its entirety). The detectable compound is not a nonspecific amyloidphilic probe such as, for example, thioflavin T, thioflavin S and Congo red dye.

The detectable compounds used in the methods described herein are intrinsically fluorescent (i.e., the molecule itself is capable of fluorescing when excited with light of the appropriate wavelength and, therefore, does not require the use of a detectable chemical tag to fluoresce). In contrast, previous detection methods described in, e.g., U.S. Patent Application Publication Nos. 2003/01040633 and 2005/0026165, require the use of polypeptides that have been conjugated to an extrinsic fluorophore such as pyrene, which has a maximum emission wavelength in the range of 475 nm to 510 nm. These detectably labeled polypeptide probes detect the presence of a prion only after the probe has undergone a conformational change upon interaction with the altered prion protein. The probe will not fluoresce unless and until it has interacted with the prion. In contrast, the detectable compounds used in the methods described herein are intrinsically fluorescent, and, therefore, do not depend on interaction with PrP-d in order to detect the presence or absence of PrP-d in a biological or environmental sample.

Detectable compounds such as Me-X04 emit at a wavelength less than 475 nm, preferably less than 460 nm and most preferably in the range of 450 nm. Other detectable compounds for use in the methods described herein are intrinsically fluorescent and emit at a wavelength greater than 600 nm, e.g., λem in the range of 600-800 nm, λem in the infrared range.

The probes utilized in the diagnostic methods specifically bind to the non-amyloid PrP-d isoform. Test samples are obtained from target tissue, e.g., a non-CNS tissue, surgically, e.g., by needle, punch biopsy or dissection, e.g., using metzenbaum scissors and forceps. The tissue is used without further dissection or the tissue is sectioned by mounting on a microscope slide. The assay is easily standardized by obtaining a reproducibly standard volume using a stylet and placing the tissue samples in a receptacle such as a 96-well microtiter plate. The tissue is dipped or bathed or otherwise contacted with a detectable non-amyloid PrP-d binding agent, washed, and fluorescence detected using a standard cuvette or microtiter plate reader. Target fluid samples and environmental samples are applied to a solid support, either directly or after enrichment or fractionation steps, and are then contacted with a detectable non-amyloid PrP-d binding agent and washed. Fluorescence is detected, e.g., using a standard cuvette, microtiter plate reader or hand-held ultraviolet fluorimeter. Target fluid samples and environmental samples are also analyzed using fluorescence polarization techniques in which the fluid sample, e.g., Thus, in fluorescence polarization, a fluid sample, such as blood, blood components, bodily fluids and other washings from fecal materials or bodily openings, e.g., the nasal cavities is, either directly or after enrichment for PrP-d, contacted with the detectable compound. The change in rotation of the compound in solution after contact with the fluid sample is then detected and analyzed.

The probes are applied to the target tissue, target fluid or environmental sample in a variety of forms, e.g., in a liquid or gel. The lipophilicity of the compounds facilitates penetration the intervening structures. The compounds bind with high avidity to accumulations of PrP-d within the target tissue, target fluid or environmental sample. For example, the compounds are formulated in a solution with an excipient, e.g., DMSO, to improve tissue and cellular penetration of the fluorescent PrP-d-binding compound. After contacting the target tissue, target tissue or environmental sample with the compound, the compound is allowed to penetrate the tissue, fluid or sample surface for a period of time, e.g., 1 minute to 5 hours, prior to fluorescent scanning of the tissue, fluid or other sample. Preferably, the tissue, fluid or environmental sample is contacted with the compound for at least one hour prior to fluorometric scanning. The tissue, fluid or sample may be contacted with the probe for up to a day or more prior to scanning. Ratiometric and other analyses of fluorophotometric signals before and after application and distribution of the fluorescent probes within a test sample reveal the degree and localization of PrP-d accumulation associated with the TSE disease state or TSE contamination. An increase in the amount of accumulated PrP-d compared to a normal control value indicates a neurodegenerative condition such as TSE or the presence of prior contamination in a sample.

Fluorescent Detection of TSE-Associated PrP-d Accumulation

The data described herein indicate that detection of non-amyloid Prp-d in target tissues and fluids such as blood and lymphatic tissue yields diagnostically-relevant information about PrP-d accumulation. A significant advantage of these methods is that they are minimally or non-invasive. The methods are also useful in in vivo drug screening, prognosing, and monitoring responses of TSE patients to therapeutic intervention.

The amount of PrP-d-binding in the target tissue, fluid or sample is monitored, e.g., by fluorophotometric techniques. Fluorescence is measured prior to application of the probe to determine baseline autofluorescence. Fluorescence is then measured again after application of the probe. The level of fluorescence before application of the probe is compared to the level after probe application. The comparison indicates the PrP-d accumulation (and a diagnosis of TSE or a predisposition to developing TSE). Binding of a lipophilic fluorescent PrP-d-binding probe, as indicated by an increased fluorescent signal in the target tissue, fluid or sample or a decrease in rotation of the fluorescence molecule in solution as measured by fluorescence polarization as compared to the level of fluorescence or level of rotation of the fluorescence molecule in tissue from a normal, healthy subject or the level of fluorescence or level of rotation of the fluorescence molecule in an uncontaminated sample, region, yields a metric which is correlated with disease presence or absence. The degree of PrP-d accumulation is greater and more rapid in infected tissues or environmental samples. This accumulation is indicative of the stage of the disease, i.e., greater accumulation is directly correlated with a more advanced stage of TSE or a related neurodegenerative state. The magnitude of fluorescence above baseline autofluorescence in the CNS correlates with disease severity. These binding data serve as a biological indicator or biomarker of PrP-d deposition within the brain.

PrP-d-specific probes are lipophilic, relatively uncharged and have a molecular weight less than 500 kDa. In contrast, detectably labeled polypeptide probes, antibody probes or antibody fragments are not suitable in the assay, because of their large molecular mass and charge. The lipophilic nature of the probes mediates efficient access to target tissues and across the lipophilic barrier of the cell membranes of the target tissues or fluids. In addition, lipophilicity facilitates access to the intracellular compartments of cells in the target tissue or target fluid.

Other probes described herein are conjugated to various small molecular fluorophors, e.g., Chrysamine G or clioquinol, using methods known in the art. For example, long wave fluorophors, e.g., Texas Red and derivatives thereof, are used. Such dyes allow scanning at wavelengths, e.g., in the far infrared range, without interference of normal target tissue or target fluid autofluorescence.

### EXAMPLE 1

### PrP-d Accumulation in TSE-Infected Ovine Subjects

As shown in FIGS. 1A and 1B, the PrP-d known as scrapie-form prion protein (PrP-d) was detected and visualized in TSE⁺ sheep. In particular, the retropharyngeal lymph nodes of the TSE⁺ sheep were analyzed ex vivo. TSE^{±} tissue was immersed in the PrP-d binding agent Me-X04 (100 μM, 10 min, RT), differentiated (alkaline EtOH 50%, pH 10), rinsed, and imaged (Optiscan 2400, Neuroptix, operating in fluorescence imaging mode with optical bandpass filters centered on excitation wavelength (λex) of approximately 370 nm, emission wavelength (λex) of approximately 450 nm). The exposures (500 msec) and post-exposure thresholding were identical. TSE status was confirmed by PrP-d immunohistochemistry.

### EXAMPLE 2

### PrP-d Accumulation in TSE-Infected Cervine Subjects

The presence of PrP-d is used as a marker for transmissible spongiform encephalopathy or a pre-morbid neurodegenerative state. The direct staining methods described herein are used to analyze tissue samples derived from the retropharyngeal lymph nodes of normal, control deer (i.e., deer that are known to be free from TSE infection) and tissue samples derived from the retropharyngeal lymph nodes of test subjects, i.e., deer suspected of being exposed to TSE. First, the retropharyngeal lymph node tissue is dissected and weighed. The control and test tissue sample are then placed in labeled quartz cuvettes and imaged. Each tissue sample is then removed from its cuvette and placed in 100 μM solution of MeXO4 in 40% ethanol/60% distilled water (adjusted to pH 10 with NaOH) for approximately 10 minutes. The tissue samples are dipped briefly in water, e.g., five times, to remove any unbound agent. The washed samples are then differentiated in a solution of 0.2% NaOH in 80% ethanol for approximately 2 minutes. The tissue samples are then placed in water for approximately 10 minutes. The samples, or thin slices thereof, are then placed on an examining surface, e.g., a glass slide, and can be covered, e.g., with fluoromount-G. The level of fluorescence in each sample is then imaged and measured. This level of fluorescence is compared to the images taken prior to contacting the tissue samples with the Me-X04 detectable agent. An increase in fluorescence in the tissue sample of the test subject, as compared to the level of fluorescence in the tissue sample from the control subject, indicates the test subject is suffering from or at risk of developing a transmissible spongiform encephalopathy.

All publications and patent documents cited herein are incorporated herein by reference as if each such publication or document was specifically and individually indicated to be incorporated herein by reference.

Other embodiments are within the following claims.

What is claimed is:

1. A method for diagnosing a Transmissible Spongiform Encephalopathy (TSE) in a mammal comprising the steps of:
(a) contacting a non-central nervous system (CNS) target tissue-derived or target fluid-derived sample with a non-peptide detectable agent that binds to a non-amyloid isoform of a disease specific prion (PrP-d), wherein the non-peptide detectable agent has a molecular weight of less than 500 kDa;
(b) imaging the sample to determine a level of fluorescence or to determine rotation of said detectable agent in said sample, wherein the level of fluorescence or rotation of the detectable agent is indicative of binding of the detectable agent to a non-amyloid isoform of PrP-d located in said sample,
wherein an increase in binding of the detectable agent to the sample compared to a normal control level of binding indicates the mammal is suffering from or at risk of developing a Transmissible Spongiform Encephalopathy.

2. The method of claim 1, the detectable binding agent is Me-X04 (1,4-bis (4′-hydroxystyrl)-2-methoxybenzene), X34 (1,4-bis(3-carboxy-4-hydroxyphenylethenyl)-benzene), Chrysamine G, or a Chrysamine G derivative.

3. The method of claim 1, wherein said method is conducted ante-mortem, post-mortem or both ante-mortem and post-mortem.

4. The method of claim 1, wherein said mammal is a bovine, a sheep, a rodent, a mink, a cervid or a human subject.

5. The method of claim 1, wherein said target tissue-derived or target fluid-derived sample is selected from the group consisting of blood, serum, eye tissue, lymphatic tissue, lymphatic cells, saliva, urine and nictitating membrane tissue.

6. The method of claim 1, wherein target tissue-derived or target fluid-derived sample is a non-ocular tissue.

7. A method for diagnosing a Transmissible Spongiform Encephalopathy (TSE) in a mammal comprising the steps of:
(a) contacting a lymphatic tissue-derived sample with a non-peptide detectable agent that binds to a disease specific prion (PrP-d);
(b) allowing said agent to distribute into the sample; and
(c) imaging the sample to determine a level of fluorescence in said sample, wherein the level of fluorescence is indicative of binding of the detectable agent to PrP-d located in said sample,
wherein an increase in binding of the detectable agent to the sample compared to a normal control level of binding indicates the mammal is suffering from a Transmissible Spongiform Encephalopathy.

8. The method of claim 10, the detectable binding agent is Me-X04 (1,4-bis (4′-hydroxystyrl)-2-methoxybenzene), Chrysamine G or a derivative thereof.

9. The method of claim 8, wherein said lymphatic tissue is peri-ocular lymphatic tissue.

10. The method of claim 8, wherein said lymphatic tissue is from a retropharyngeal lymphatic tissue, an oral lymphatic tissue or a peri-oral lymphatic tissue.

11. The method of claim 8, wherein said method is conducted ante-mortem, post-mortem or both ante-mortem and post-mortem.

12. The method of claim 8, wherein said mammal is a bovine, a sheep, a rodent, a mink, a cervid or a human.

13. A method for diagnosing a Transmissible Spongiform Encephalopathy (TSE) in a mammal comprising the steps of:
(a) contacting a cerebrospinal fluid (CSF)-derived sample with a non-peptide detectable non-peptide agent that binds to a disease specific prion (PrP-d);
(b) allowing said agent to distribute into the sample; and
(c) imaging the sample to determine a level of fluorescence or to determine rotation of said detectable agent in said sample, wherein the level of fluorescence or rotation of said detectable agent is indicative of binding of the detectable agent to PrP-d located in said sample,
wherein an increase in binding of the detectable agent to the sample compared to a normal control level of binding indicates the mammal is suffering from a Transmissible Spongiform Encephalopathy.

14. The method of claim 13, the detectable binding agent is Me-X04 (1,4-bis (4′-hydroxystyrl)-2-methoxybenzene), X34 (1,4-bis(3-carboxy-4-hydroxyphenylethenyl)-benzene), Chrysamine G, or a Chrysamine G derivative.

15. The method of claim 13, wherein said method is conducted ante-mortem, post-mortem or both ante-mortem and post-mortem.

16. The method of claim 13, wherein said mammal is a bovine, a sheep, a rodent, a mink, a cervid or a human.

17. A method for diagnosing a Transmissible Spongiform Encephalopathy (TSE) in a mammal comprising the steps of:
(a) contacting a target tissue-derived sample, a target fluid-derived sample or environmental sample with a detectable agent that preferentially binds to a disease specific prion (PrP-d), wherein the agent is not an antibody;
(b) allowing said agent to distribute into the target tissue-derived sample, the target fluid-derived sample or environmental sample; and
(c) imaging the target tissue-derived sample, the target fluid-derived sample or environmental sample to determine a level of fluorescence or to determine rotation of said detectable agent in said target tissue-derived sample, target fluid-derived sample or environmental sample, wherein the level of fluorescence or rotation of said detectable agent is indicative of binding of the detectable agent to PrP-d located in said target tissue-derived sample, target fluid-derived sample or environmental sample,
wherein an increase in binding of the detectable agent to the target tissue-derived sample, the target fluid-derived sample or environmental sample compared to a normal control level of binding indicates infection of said mammal or contamination of said environmental sample with an infectious agent of Transmissible Spongiform Encephalopathy.

18. The method of claim 17, the detectable binding agent is Me-X04 (1,4-bis (4′-hydroxystyrl)-2-methoxybenzene), X34 (1,4-bis(3-carboxy-4-hydroxyphenylethenyl)-benzene), Chrysamine G, or a Chrysamine G derivative.

19. The method of claim 17, wherein said method is conducted ante-mortem, post-mortem or both ante-mortem and post-mortem.

20. The method of claim 17, wherein said mammal is a bovine, a sheep, a rodent, a mink, a cervid or a human.

21. The method of claim 17, wherein said target tissue-derived or target fluid-derived sample is selected from the group consisting of blood, eye tissue, lymphatic tissue, saliva, urine, cerebrospinal fluid, and nictitating membrane tissue.

22. The method of claim 17, wherein said environmental sample is a land material sample, a water sample, a surgical surface, a surgical instrument, slaughtering surface, a butchering surface, or any combination thereof.

23. A method for detecting the presence of an infectious agent of Transmissible Spongiform Encephalopathy (TSE) in a non-mammalian test sample comprising contacting said sample with a detectable agent that binds to a non-amyloid isoform of a disease specific prion (PrP-d), wherein an increase in binding of the detectable agent to the target tissue compared to a normal control level of binding indicates that the sample is contaminated with an infectious agent a Transmissible Spongiform Encephalopathy.

24. The method of claim 23, wherein the sample is selected from an environmental sample, a surgical surface, a surgical instrument, a slaughtering surface, and a butchering surface.
